(19) United States
(12) Patent Application Publication
(10) Pub. No.: US 2024/0026278 A1
(43) Pub. Date: Jan. 25, 2024

(54) BLOOD CELL LYSING AGENT FOR ISOLATING BACTERIA FROM BLOOD CULTURE

(71) Applicant: Becton, Dickinson and Company, Franklin Lakes, NJ (US)

(72) Inventors: Christopher MASSEY, Stewartstown, PA (US); Jindong ZAN, Timonium, MD (US); Axel A. YUP, Owings Mills, MD (US)

(21) Appl. No.: 18/041,975

(22) PCT Filed: Aug. 19, 2021
(86) PCT No.: PCT/US2021/046750

§ 371 Date: Feb. 16, 2023

Related U.S. Application Data
(60) Provisional application No. 63/068,278, filed on Aug. 20, 2020.

(51) Int. Cl. C12N 1/06; C12Q 1/24; C12Q 1/04

(52) U.S. Cl. CPC C12N 1/06; C12Q 1/24; C12Q 1/04

(57) ABSTRACT

Disclosed herein include methods, compositions, and kits suitable for use in processing a sample comprising blood cells and at least one microorganism. In some embodiments, the method comprises contacting the sample with a lysis buffer to generate a treated sample. The lysis buffer can comprise a Somatic Cell Digestion Agent (SDA) capable of lysing blood cells in the sample. In some embodiments, the at least one microorganism remains intact and/or viable in the presence of the SDA.

### RELATED APPLICATIONS

The present application claims priority under 35 U.S.C. § 119(e) to U.S. Provisional Application No. 63/068,278, filed Aug. 20, 2020. The entire contents of these applications are hereby expressly incorporated by reference in their entireties.

### BACKGROUND

### Field

The present disclosure relates generally to the field of microbial isolation and identification.

### Description of the Related Art

Sepsis is a serious medical condition caused by an overwhelming response of the host immune system to infection. It can trigger widespread inflammation, which can give rise to impaired blood flow. As sepsis progresses, the body's organs can be starved for oxygen and nutrients, causing permanent damage and eventual failure. Left improperly diagnosed or otherwise untreated, the heart may weaken and septic shock can occur, leading to multiple organ failure and death. Blood cultures are required to detect the presence of bacteria or yeast in the blood of sepsis patients. If a microorganism is present, (positive blood culture (“PBC”)) the microorganism(s) must be identified and antibiotic susceptibility determined in order to provide appropriate treatment. The PBC samples are used to isolate, identify and perform antimicrobial susceptibility testing (“AST”). The microorganism(s) are often identified by methods such as mass spectrometry, including MALDI-TOF/MS or phenotypic growth-based methods, such as Phoenix™ ID.

In order to identify the microorganism(s), perform phenotypic analysis on the microorganism, and perform AST testing, intact, and/or viable microorganism(s) need(s) to be isolated from the blood cells and other material in the collected sample. For identification of the microorganism by mass spectrometry, the microbial sample needs to be sufficiently free from substances known to interfere with MALDI-TOF/MS identification, such as blood cell components, other cellular debris, and salts. In addition, the microbial sample needs to be of sufficient quantity in order to obtain a reliable identification. Phenotypic identification methods, such as Phoenix™ ID, require intact, viable microorganism free from substances that may interfere with the enzymatic substrates of the assay. For AST testing, such as Phoenix™ AST, the microbial sample needs to contain viable, unaltered microorganism capable of growth in the presence of antibiotic, if resistance mechanisms are present, during performance of the assay. It is important for all methods to be of sufficient quantity and purity as carryover of residual blood or media components will interfere either directly or by falsely increasing the concentration (turbidity) of microorganism.

Current techniques for isolating viable microorganism from a PBC sample include sub-culturing the microorganism(s), which can take up to 72 hours. This results in the delay of treatment or treatment with inappropriate antibiotics.

Certain strains of microorganisms are particularly difficult to isolate from a PBC sample while maintaining viability of the organism, such as, for example, Streptococcus pneumoniae (S. pneumoniae). Part of this difficulty is traced to the activation of autolysin by S. pneumoniae which causes the microbial cells to “self-destruct”. See “Streptococcus pneumoniae Antigen Test Using Positive Blood Culture Bottles as an Alternative Method To Diagnose Pneumococcal Bacteremia”, Journal of Clinical Microbiology, Vol. 43, No. 5, May 2005, pp. 2510-2512. The current method for isolating microorganisms from septic patients, including, S. pneumoniae, includes inoculating blood culture bottles. Once a positive signal is achieved, a portion of the PBC sample is removed to perform a gram stain and another portion is used to sub-culture the microorganism. Microbial colonies from the sub-culture are used to perform downstream testing such as identification by MALDI-TOF/MS, phenotypic identification methods, and AST testing.

Additional techniques for isolating viable microorganism(s) from a PBC sample often utilize liquid separation methods containing lysis buffers with detergents that lyse the blood cells in the PBC sample. After lysis, the lysed blood cells can be removed while the microorganism(s) is/are retained. However, the use of these lysis buffers often result in compromised, damaged, or non-viable microorganism(s) which is/are insufficient for performing certain growth-based identification methods such as AST testing.

Currently available sample processing methods and compositions suffer from various deficiencies, such as (i) insufficient viability after sample processing to support growth-based identification methods and AST methods, due to the interaction of the harsh detergents on the microbial cell wall; (ii) produce inconsistent identification of the microorganism at the species level across a panel of microorganisms; and/or (iii) do not allow for the isolation of viable microorganism from a PBC sample that is free from interfering substances and would allow for multiple downstream testing from one PBC sample, such as both MALDI-TOF/MS identification and AST testing.

Accordingly, there is a need for efficient blood cell lysing agents for isolating microorganisms from positive blood cultures for rapid MALDI identification. There is a need for blood cell lysing agents for identification of challenging species such as Staphylococcus epidermidis that generate low MALDI scores when isolated form positive blood cultures using currently available methods.

### SUMMARY

Disclosed herein include methods of processing a sample. The method can comprises: contacting a sample comprising blood cells and at least one microorganism with a lysis buffer to generate a treated sample, wherein the lysis buffer comprises a Somatic Cell Digestion Agent (SDA) capable of lysing blood cells in the sample, wherein the SDA is a compound of Formula 1,

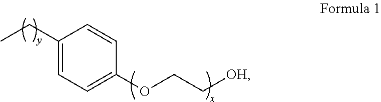

wherein x is an integer from 2 to 20, and wherein y is an integer from 6 to 11, thereby lysing the blood cells in the sample. In some embodiments, y is an integer from 8 to 10. In some embodiments, y is 8. In some embodiments, x is an integer from 5 to 15. In some embodiments, x is an integer from 8 to 12, for example 9 or In some embodiments, x is 9. In some embodiments, the SDA is Nonoxynol-9.

In some embodiments, the concentration of the SDA in the lysis buffer is about g/L to about 10 g/L. In some embodiments, the concentration of the SDA in the lysis buffer is about 0.01% (w/w) to about 10% (w/w). In some embodiments, the concentration of the SDA in the lysis buffer is about 0.01% (w/w) to about 1% (w/w). In some embodiments, the concentration of the SDA in the lysis buffer is about 0.52% (w/w).

In some embodiments, the sample is derived from a blood culture of a subject suspected of having an infection. In some embodiments, the sample comprises a positive blood culture sample determined to comprise at least one microorganism therein. In some embodiments, the at least one microorganism is selected from the group comprising gram-positive bacteria, gram-negative bacteria, and yeast. In some embodiments, the at least one microorganism is S. epidermidis. In some embodiments, the at least one microorganism comprises one or more of Enterococcus faecalis, Pseudomonas aeruginosa, E. coli, and S. pneumoniae.

In some embodiments, the contacting step comprises sonication, osmotic shock, chemical treatment, or any combination thereof. In some embodiments, the lysis buffer comprises one or more proteinases and/or one or more nucleases. The method can comprise: isolating the at least one microorganism from the treated sample to generate at least one isolated microorganism. In some embodiments, isolating the at least one microorganism from the treated sample comprises separating the at least one microorganism from lysed blood cells. In some embodiments, separating the at least one microorganism from lysed blood cells comprises: centrifuging the treated sample to produce a pellet and a supernatant; and discarding the supernatant while retaining the pellet comprising at least one isolated microorganism. The method can comprise: preparing a plated pure culture from the at least one isolated microorganism and analyzing the microorganism obtained from the plated pure culture. The method can comprise: preparing an inoculum from the at least one isolated microorganism and analyzing the at least one microorganism obtained from the inoculum.

The method can comprise: depositing at least a portion of the pellet comprising at least one isolated microorganism on a surface adapted to be placed in an apparatus configured to determine the identity of the at least one microorganism by mass spectrometry; optionally, drying the deposited sample; treating the deposited sample with a volatile acid solution, wherein the volume percent of the volatile acid is at least 70% of the volatile acid solution combined with the deposited sample; optionally, drying the treated deposited sample; placing a matrix over the treated deposited sample; and optionally, drying the treated deposited sample. In some embodiments, the volatile acid solution is a volatile acid in water or a volatile solution in an organic solvent. In some embodiments, the volatile acid solution is formic acid in water at a volume percent of 70% when combined with the deposited sample. In some embodiments, the volatile acid solution is formic acid in water at a volume percent of about 80% when combined with the deposited sample. In some embodiments, the volatile acid solution is formic acid in water at a volume percent of about 90% when combined with the deposited sample. The method can comprise, prior to treating the deposited sample with a volatile acid solution, treating the deposited sample with an organic solvent and drying the deposited sample. In some embodiments, the organic solvent comprises ethanol, methanol, isopropanol, acetonitrile, acetone, ethyl acetate, or any combination thereof.

The method can comprise: contacting the sample with a choline-containing solution before, simultaneously, and/or after contacting the sample with the lysis buffer. In some embodiments, the choline-containing solution comprises at least one quarternary ammonium salt containing a N,N,N-trimethylethanolammonium cation selected from the group consisting of Formula 2,

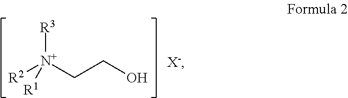

wherein R¹, R², and R³ independently represent one selected from the group consisting of a saturated hydrocarbon group, an unsaturated hydrocarbon group, an aromatic group, and combinations thereof, and wherein X represents a negative charged group. In some embodiments, X is selected from the group consisting of chloride, fluoride, nitrate, and bicarbonate. In some embodiments, the choline-containing solution comprises choline chloride. In some embodiments, the choline-containing solution comprises phosphorylcholine. In some embodiments, the final concentration of choline when contacted with the sample is greater than or equal to about 0.25% by volume. In some embodiments, the final concentration of choline when contacted with the sample is greater than or equal to about 1% by volume. In some embodiments, the concentration of choline in the sample during the contacting is about 1.8% by volume. In some embodiments, the concentration of choline in the sample during the contacting is about 4% by volume. In some embodiments, the concentration of choline in the sample during the contacting is in the range of about 0.25% by volume to about 10% by volume. In some embodiments, the contacting comprises incubating the sample with the choline-containing solution for up to 20 minutes, and the temperature of said incubation is room temperature.

The lysis buffer can further comprises an antifoaming agent. In some embodiments, the lysis buffer does not comprise an antifoaming agent. In some embodiments, the lysis buffer further comprises at least one thiol. In some embodiments, the at least one thiol comprises L-cysteine HCL, sodium thioglycolate, mercaptoethylamine, mercaptosuccinic acid, mercaptoethanol, mercaptoethane sulfonic acid, thioglycerol, or any combination thereof, optionally the concentration of the at least one thiol in the lysis buffer is about 0.005 g/L to 4 g/L. In some embodiments, the at least one thiol comprises L-cysteine at a concentration in the lysis buffer of about 0.01 g/L to about 2.5 g/L, and/or sodium thioglycolate at a concentration in the lysis buffer of about 0.01 g/L to about 2.5 g/L. In some embodiments, the lysis buffer further comprises ammonium chloride, wherein the concentration of ammonium chloride in the lysis buffer is about 0.01 g/L to about 80 g/L. In some embodiments, the lysis buffer further comprises a nutrient base solution comprising one or more of casein peptone at a concentration in the lysis buffer of about 8 g/L to about 35 g/L, sodium chloride at a concentration in the lysis buffer of about 2 g/L to about 10 g/L, soy peptone at a concentration in the lysis buffer of about 1.5 g/L to about 15 g/L, potassium phosphate at a concentration in the lysis buffer of about 0.5 g/L to about 5 g/L, and at least one other nutrient. In some embodiments, the at least one other nutrient comprises a nutrient broth at a concentration in the lysis buffer of about 10 g/L to about 50 g/L. In some embodiments, the at least one other nutrient comprises a nutrient broth comprising one or more of: i) tryptone; ii) soy; iii) NaCl; iv) dipotassium phosphate (K₂HPO₄); and v) glucose.

In some embodiments, the lysis buffer further comprises one or more of a nutrient broth, an isotonic buffer, a peptone, and a salt, optionally the concentration of the nutrient broth in the lysis buffer is about 10 g/L to about 50 g/L. In some embodiments, the nutrient broth comprises trypticase soy broth. In some embodiments, the isotonic buffer comprises sodium phosphate, potassium phosphate, phosphate buffered saline, saline, or any combination thereof, optionally the concentration of isotonic buffer in the lysis buffer is about 1 g/L to about 20 g/L. In some embodiments, the peptone comprises casein peptone and/or soy peptone. In some embodiments, the lysis buffer further comprises sodium pyruvate, yeast extract, sodium citrate, meat peptones, dextrose, phosphate buffered saline, or any combination thereof. In some embodiments, the lysis buffer further comprises at least one additional non-ionic detergent, optionally the at least one additional non-ionic detergent comprises saponin. In some embodiments, the lysis buffer does not comprise an additional non-ionic detergent.

The method can comprise: identifying the at least one microorganism. In some embodiments, identifying the at least one microorganism comprises mass spectrometry, phenotypic identification, antimicrobial susceptibility testing, molecular testing, or any combination thereof. In some embodiments, mass spectrometry comprises one or more of electrospray ionization mass spectrometry (ESI-MS), ESI-MS/MS, ESI-MS/(MS)ⁿ, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), APCJ-MS/MS, APCI-(MS)ⁿ, atmospheric pressure photoionization mass spectrometry (APPI-MS), APPI-MS/MS, and APPI-(MS)ⁿ, quadrupole mass spectrometry, Fourier transform mass spectrometry (FTMS), and ion trap mass spectrometry, where n is an integer greater than zero. In some embodiments, mass spectrometry comprises MALDI-TOF-MS.

In some embodiments, the SDA does not damage the at least one microorganism. For example, the at least one microorganism can remain intact and/or viable in the presence of the SDA. In some embodiments, the method yields an at least 5% higher MALDI score as compared to a comparable method employing a lysis buffer that does not comprise the SDA. In some embodiments, the comparable method employs a lysis buffer comprising saponin. In some embodiments, the lysis buffer selectively lyses at least about 1%, at least about 5%, at least about 10%, at least about 20%, at least about 30%, at least about 40%, at least about 50%, at least about 60%, at least about 70%, at least about 80%, at least about 90%, at least about 95%, or at least about 99%, of the blood cells in the sample. In some embodiments, the ratio of blood cells lysed to cells of the at least one microorganism lysed following the contacting step is at least about 2:1. In some embodiments, at least about 50%, at least about 60%, at least about 70%, at least about 80%, at least about 90%, at least about 95%, or at least about 99%, of the cells of the at least one microorganism remain intact and/or viable following the contacting step.

In some embodiments, the lysis buffer does not comprise a buffering agent. In some embodiments, the lysis buffer is acidic. In some embodiments, identifying the at least one microorganism does not comprise spectroscopy, e.g., intrinsic fluorescence spectroscopy. In some embodiments, the method does not comprise density gradient centrifugation. In some embodiments, the lysis buffer does not comprise saponin. In some embodiments, the lysis buffer does not comprise one or more detergents selected from the group consisting of Triton® X-100, Triton® X-100-R, Triton® X-114, NP-40, Genapol® C-100, Genapol® X-100, Igepal® CA 630, Arlasolve™200, Brij® 96/97, CHAPS, octyl β-D-glucopyranoside, saponin, nonaethylene glycol monododecyl ether (C12E9, polidocenol), sodium dodecyl sulfate, N-laurylsarcosine, sodium deoxycholate, bile salts, hexadecyltrimethylammonium bromide, SB3-10, SB3-12, amidosulfobetaine-14, C7BzO, Brij® 98, Brij® 58, Brij® 35, Tween® 80, Tween® 20, Pluronic® L64, Pluronic® P84, non-detergent sulfobetaines (NDSB 201), amphipols (PMAL-C8), and methyl-β-cyclodextrin. In some embodiments, the lysis buffer does not comprise one or more detergents selected from the group consisting of Triton® X-100, Triton® X-100-R, Triton® X-114, NP-40, Igepal CA 630, Arlasolve 200, Brij® 96/97, CHAPS, octyl β-D-glucopyranoside, saponin, nonaethylene glycol monododecyl ether. In some embodiments, the lysis buffer does not comprise one or more detergents selected from the group consisting of sodium dodecyl sulfate, N-laurylsarcosine, sodium dexoychloate, bile salts, hexadecyltrimethylammonium bromide, SB3-10, SB3-12, amidosulfobetaine-14, C₇BzO. In some embodiments, the lysis buffer does not comprise one or more detergents selected from the group consisting of Brij® 97, Brij® 96V, Genapol® C-100, Genapol® X-100, and polidocenol. In some embodiments, the lysis buffer does not comprise a polyoxyethylene detergent comprising the structure C₁₂₋₁₈/E₉₋₁₀, wherein C₁₂₋₁₈ denotes a carbon chain length of from 12 to 18 carbon atoms and E₉₋₁₀ denotes from 9 to 10 oxyethylene hydrophilic head groups.

Disclosed herein include compositions (e.g., kits). In some embodiments, composition comprises: a lysis buffer comprising a Somatic Cell Digestion Agent (SDA) capable of lysing blood cells, wherein the SDA is a compound of Formula 1,

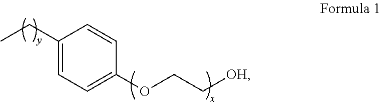

wherein x is an integer from 2 to 20, and wherein y is an integer from 6 to 11; and blood cells and/or debris thereof. In some embodiments, y is an integer from 8 to 10. In some embodiments, y is 8. In some embodiments, x is an integer from 5 to 15. In some embodiments, x is an integer from 8 to 12. In some embodiments, x is 9 or In some embodiments, x is 9. In some embodiments, the SDA is Nonoxynol-9.

In some embodiments, the concentration of the SDA in the lysis buffer is about g/L to about 10 g/L. In some embodiments, the concentration of the SDA in the lysis buffer is about 0.01% (w/w) to about 10% (w/w), e.g., about 0.01% (w/w) to about 1% (w/w). In some embodiments, the concentration of the SDA in the lysis buffer is about 0.52% (w/w). In some embodiments, the lysis buffer comprises one or more proteinases and/or one or more nucleases.

In some embodiments, the composition comprises a choline-containing solution comprising at least one quarternary ammonium salt containing a N,N,N-trimethylethanolammonium cation selected from the group consisting of Formula 2,

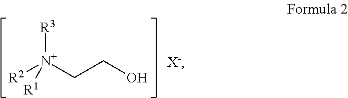

wherein R¹, R², and R³ independently represent one selected from the group consisting of a saturated hydrocarbon group, an unsaturated hydrocarbon group, an aromatic group, and combinations thereof, and wherein X represents a negative charged group. In some embodiments, X is selected from the group consisting of chloride, fluoride, nitrate, and bicarbonate. In some embodiments, the choline-containing solution comprises choline chloride. In some embodiments, the choline-containing solution comprises phosphorylcholine.

In some embodiments, the lysis buffer further comprises an antifoaming agent. In some embodiments, the lysis buffer does not comprise an antifoaming agent. In some embodiments, the lysis buffer further comprises at least one thiol. In some embodiments, the at least one thiol comprises L-cysteine HCL, sodium thioglycolate, mercaptoethylamine, mercaptosuccinic acid, mercaptoethanol, mercaptoethane sulfonic acid, thioglycerol, or any combination thereof, optionally the concentration of the at least one thiol in the lysis buffer is about 0.005 g/L to 4 g/L. In some embodiments, the at least one thiol comprises L-cysteine at a concentration in the lysis buffer of about 0.01 g/L to about 2.5 g/L, and/or sodium thioglycolate at a concentration in the lysis buffer of about 0.01 g/L to about 2.5 g/L. In some embodiments, the lysis buffer further comprises ammonium chloride, the concentration of ammonium chloride in the lysis buffer is about 0.01 g/L to about 80 g/L. In some embodiments, the lysis buffer further comprises a nutrient base solution comprising one or more of casein peptone at a concentration in the lysis buffer of about 8 g/L to about 35 g/L, sodium chloride at a concentration in the lysis buffer of about 2 g/L to about 10 g/L, soy peptone at a concentration in the lysis buffer of about 1.5 g/L to about 15 g/L, potassium phosphate at a concentration in the lysis buffer of about 0.5 g/L to about 5 g/L, and at least one other nutrient. In some embodiments, the at least one other nutrient comprises a nutrient broth at a concentration in the lysis buffer of about 10 g/L to about 50 g/L. In some embodiments, the at least one other nutrient comprises a nutrient broth comprising one or more of: i) tryptone; ii) soy; iii) NaCl; iv) dipotassium phosphate (K₂HPO₄); and v) glucose.

In some embodiments, the lysis buffer further comprises one or more of a nutrient broth, an isotonic buffer, a peptone, and a salt, optionally the concentration of the nutrient broth in the lysis buffer is about 10 g/L to about 50 g/L. In some embodiments, the nutrient broth comprises trypticase soy broth. In some embodiments, the isotonic buffer comprises sodium phosphate, potassium phosphate, phosphate buffered saline, saline, or any combination thereof, optionally the concentration of isotonic buffer in the lysis buffer is about 1 g/L to about 20 g/L. In some embodiments, the peptone comprises casein peptone and/or soy peptone. In some embodiments, the lysis buffer further comprises sodium pyruvate, yeast extract, sodium citrate, meat peptones, dextrose, phosphate buffered saline, or any combination thereof. In some embodiments, the lysis buffer further comprises at least one additional non-ionic detergent, optionally the at least one additional non-ionic detergent comprises saponin.

In some embodiments, the lysis buffer does not comprise an additional non-ionic detergent. In some embodiments, the lysis buffer does not comprise a buffering agent. In some embodiments, the lysis buffer is acidic. In some embodiments, the lysis buffer does not comprise saponin. In some embodiments, the lysis buffer does not comprise one or more detergents selected from the group consisting of Triton® X-100, Triton® X-100-R, Triton® X-114, NP-40, Genapol® C-100, Genapol® X-100, Igepal® CA 630, Arlasolve™200, Brij® 96/97, CHAPS, octyl β-D-glucopyranoside, saponin, nonaethylene glycol monododecyl ether (C12E9, polidocenol), sodium dodecyl sulfate, N-laurylsarcosine, sodium deoxycholate, bile salts, hexadecyltrimethylammonium bromide, SB3-10, SB3-12, amidosulfobetaine-14, C7BzO, Brij® 98, Brij® 58, Brij® 35, Tween® 80, Tween® 20, Pluronic® L64, Pluronic® P84, non-detergent sulfobetaines (NDSB 201), amphipols (PMAL-C8), and methyl-β-cyclodextrin. In some embodiments, the lysis buffer does not comprise one or more detergents selected from the group consisting of Triton® X-100, Triton® X-100-R, Triton® X-114, NP-40, Igepal CA 630, Arlasolve 200, Brij® 96/97, CHAPS, octyl β-D-glucopyranoside, saponin, nonaethylene glycol monododecyl ether. In some embodiments, the lysis buffer does not comprise one or more detergents selected from the group consisting of sodium dodecyl sulfate, N-laurylsarcosine, sodium dexoychloate, bile salts, hexadecyltrimethylammonium bromide, SB3-10, SB3-12, amidosulfobetaine-14, C₇BzO. In some embodiments, the lysis buffer does not comprise one or more detergents selected from the group consisting of Brij® 97, Brij® 96V, Genapol® C-100, Genapol® X-100, and polidocenol. In some embodiments, the lysis buffer does not comprise a polyoxyethylene detergent comprising the structure C₁₂₋₁₈/E₉₋₁₀, wherein C₁₂₋₁₈ denotes a carbon chain length of from 12 to 18 carbon atoms and E₉₋₁₀ denotes from 9 to 10 oxyethylene hydrophilic head groups. In some embodiments, the at least one microorganism remains intact in the presence of the SDA. In some embodiments, the SDA does not damage at least one microorganism.

### BRIEF DESCRIPTION OF THE DRAWINGS

FIG. 1 depicts exemplary data related to MALDI scores for Staphylococcus epidermidis isolated from positive blood cultures using different lysing agents. MALDI scores for Staphylococcus epidermidis isolated from positive blood cultures using different lysing agents are shown. SAP stands for Saponin, SDA stands for Somatic Cell Digestion Agent Nonoxynol-9. The concentration of each lysing agent is used at 0.52% (W/W). The score for acceptance of identification is greater than or equal to 1.8 for Sepsityper database and 2.0 for standard database.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods of processing a sample. In some embodiments, the method comprises: contacting a sample comprising blood cells and at least one microorganism with a lysis buffer to generate a treated sample, wherein the lysis buffer comprises a Somatic Cell Digestion Agent (SDA) capable of lysing blood cells in the sample, wherein the SDA is a compound of Formula 1,

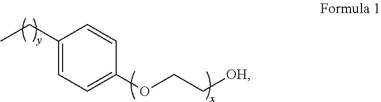

wherein x is an integer from 2 to 20, and wherein y is an integer from 6 to 11, thereby lysing the blood cells in the sample. In some embodiments, y is an integer from 8 to 10. In some embodiments, y is 8. In some embodiments, x is an integer from 5 to 15. In some embodiments, x is an integer from 8 to 12. In some embodiments, x is 9 or 10. In some embodiments, x is 9. In some embodiments, the SDA is Nonoxynol-9.

Disclosed herein include compositions (e.g., kits). In some embodiments, composition comprises: a lysis buffer comprising a Somatic Cell Digestion Agent (SDA) capable of lysing blood cells, wherein the SDA is a compound of Formula 1,

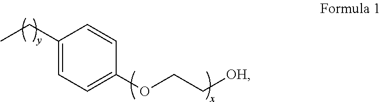

wherein x is an integer from 2 to 20, and wherein y is an integer from 6 to 11; and blood cells and/or debris thereof. In some embodiments, y is an integer from 8 to 10. In some embodiments, y is 8. In some embodiments, x is an integer from 5 to 15. In some embodiments, x is an integer from 8 to 12. In some embodiments, x is 9 or 10. In some embodiments, x is 9. In some embodiments, the SDA is Nonoxynol-9.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, N Y 1989). For purposes of the present disclosure, the following terms are defined below.

As used herein, the term “about,” when referring to a measurable value such as an amount of a compound or agent disclosed herein (e.g., SDA), dose, time, temperature, and the like, shall be given its ordinary meaning and shall also encompass variations of ±20%, ±10%, ±5%, ±1%, ±0.5%, or even ±0.1% of the specified amount.

As used herein, the term “microorganism” shall be given its ordinary meaning and shall also refer to organisms that are generally unicellular, which can be multiplied and handled in the laboratory, including but not limited to, Gram-positive or Gram-negative bacteria, yeasts, molds, parasites, and mollicutes. Non-limiting examples of Gram-negative bacteria include bacteria of the following genera: Pseudomonas, Escherichia, Salmonella, Shigella, Enterobacter, Klebsiella, Serratia, Proteus, Campylobacter, Haemophilus, Morganella, Vibrio, Yersinia, Acinetobacter, Stenotrophomonas, Brevundimonas, Ralstonia, Achromobacter, Fusobacterium, Prevotella, Branhamella, Neisseria, Burkholderia, Citrobacter, Hafnia, Edwardsiella, Aeromonas, Moraxella, Brucella, Pasteurella, Providencia, and Legionella. Non-limiting examples of Gram-positive bacteria include bacteria of the following genera: Enterococcus, Streptococcus, Staphylococcus, Bacillus, Paenibacillus, Lactobacillus, Listeria, Peptostreptococcus, Propionibacterium, Clostridium, Bacteroides, Gardnerella, Kocuria, Lactococcus, Leuconostoc, Micrococcus, Mycobacteria and Corynebacteria. Non-limiting examples of yeasts and molds include those of the following genera: Candida, Cryptococcus, Nocardia, Penicillium, Alternaria, Rhodotorula, Aspergillus, Fusarium, Saccharomyces and Trichosporon. Non-limiting examples of parasites include those of the following genera: Trypanosoma, Babesia, Leishmania, Plasmodium, Wucheria, Brugia, Onchocerca, and Naegleria. Non-limiting examples of mollicutes include those of the following genera: Mycoplasma and Ureaplasma.

In some embodiments of the methods and compositions disclosed herein, microorganisms from a sample or growth medium can be separated and interrogated to characterize and/or identify the microorganism present in the sample. As used herein, the term “separate” shall be given its ordinary meaning and shall also encompass any sample of microorganisms that has been removed, concentrated or otherwise set apart from its original state, or from a growth or culture medium. For example, in some embodiments, microorganisms may be separated away (e.g., as a separated sample) from non-microorganisms or non-microorganism components that may otherwise interfere with characterization and/or identification. A separated microorganism sample can include any collection or layer of microorganisms and/or components thereof that is more concentrated than, or otherwise set apart from, the original sample, and can range from a closely packed dense clump of microorganisms to a diffuse layer of microorganisms. Microorganism components that can be comprised in a separated form or sample include, without limitation, pilli, flagella, fimbriae, and capsules in any combination. Non-microorganism components that are separated away from the microorganisms may include non-microorganism cells (e.g., blood cells and/or other tissue cells) and/or any components thereof.

In some embodiments of the methods and compositions disclosed herein, microorganisms from a sample or growth medium can be isolated and interrogated to characterize and/or identify the microorganism present in the sample. As used herein, the term “isolated” shall be given its ordinary meaning and shall also encompass any sample of microorganisms that has been at least partially purified from its original state, or from a growth or culture medium, and any non-microorganisms or non-microorganism components contained therein. For example, in some embodiments, microorganisms are isolated away (e.g., as an isolated sample) from non-microorganisms or non-microorganism components that may otherwise interfere with characterization and/or identification. Non-microorganism components that are separated away from the microorganisms can include non-microorganism cells (e.g., blood cells and/or other tissue cells) and/or any components thereof.

In some embodiments of the methods and compositions disclosed herein, microorganisms from a sample or growth medium can be pelleted and interrogated to characterize and/or identify the microorganism present in the sample. As used herein, the term “pellet” shall be given its ordinary meaning and shall also encompass any sample of microorganisms that has been compressed or deposited into a mass of microorganisms. For example, microorganisms from a sample can be compressed or deposited into a mass at the bottom of a tube by centrifugation, or other known methods in the art. The term includes a collection of microorganisms (and/or components thereof) on the bottom and/or sides of a container following centrifugation. Microorganism components that can be comprised in a pellet include, without limitation, pilli, flagella, fimbriae, and capsules in any combination. In some embodiments, microorganisms may be pelleted away (e.g., as a substantially purified microorganism pellet) from non-microorganisms or non-microorganism components that may otherwise interfere with characterization and/or identification. Non-microorganism components that are separated away from the microorganisms may include non-microorganism cells (e.g., blood cells and/or other tissue cells) and/or any components thereof.

### Lysis Buffers and Methods of Using

Various embodiments disclosed herein provide for reagents and methods for rapidly isolating intact and/or viable microbial cells from a sample (e.g., PBC) including S. epidermidis. The resulting microbial pellet obtained using the various disclosed reagents and methods can be sufficiently free from interfering substances and can be used for identification methods, such as MALDI-TOF/MS, growth-based identification and AST methods. This can enable rapid results without the need for sub-culturing the microorganisms. The concentrated mass of viable microbial cells obtained by the various embodiments can be used for the direct inoculation of rapid ID systems, such as MALDI-TOF/MS, and ID/AST testing (AST) by conventional or automated systems, such as the BD™ Phoenix™ ID/AST system. The various embodiments can also be applicable to other systems, molecular testing methods, such as polymerase chain reaction (PCR), and other methods known to one skilled in the art.

Various embodiments disclosed herein provide for reagents and methods for rapidly isolating microbial cells, including Staphylococcus epidermidis, from positive blood culture samples. The resulting microbial pellet can be used for identification and/or growth-based methods such as antimicrobial susceptibility testing. In some embodiments, the disclosed methods provide a process for rapidly isolating and concentrating viable microorganism (s) from PBC samples using only one sample preparation tube and centrifugation while removing cellular debris from the mammalian blood cells that may interfere with identification methods. A positive blood culture (PBC) sample may be obtained by methods known to those skilled in the art and is not described in detail herein. The PBC sample may include samples determined to be positive for at least one microorganism by detection with, for example, the BD BACTEC™ Instrumented Blood Culture System (Becton, Dickinson and Company). In one embodiment the microorganism(s) includes gram-positive bacteria, gram-negative bacteria, or yeast. In some embodiments, the microorganism(s) is S. epidermidis. The starting volume of the PBC sample is not limited to any particular maximum or minimum volume.

Provided herein are methods and compositions comprising SDA (Somatic Cell Digestion Agent), a novel and efficient blood cell lysing surfactant. In some embodiments, SDA is an efficient blood cell lysing agent for isolating bacteria from positive blood cultures for rapid MALDI identification. In some embodiments, SDA is non-ionic surfactant Nonoxynol-9. In some embodiments of the compositions and methods disclosed herein, SDA is employed in a lysis buffer to lyse blood cells to facilitate isolating bacterial cells from positive blood culture for rapid identification by MALDI. In some embodiments, SDA can specifically disrupt the blood cell membrane without damaging bacterial cells.

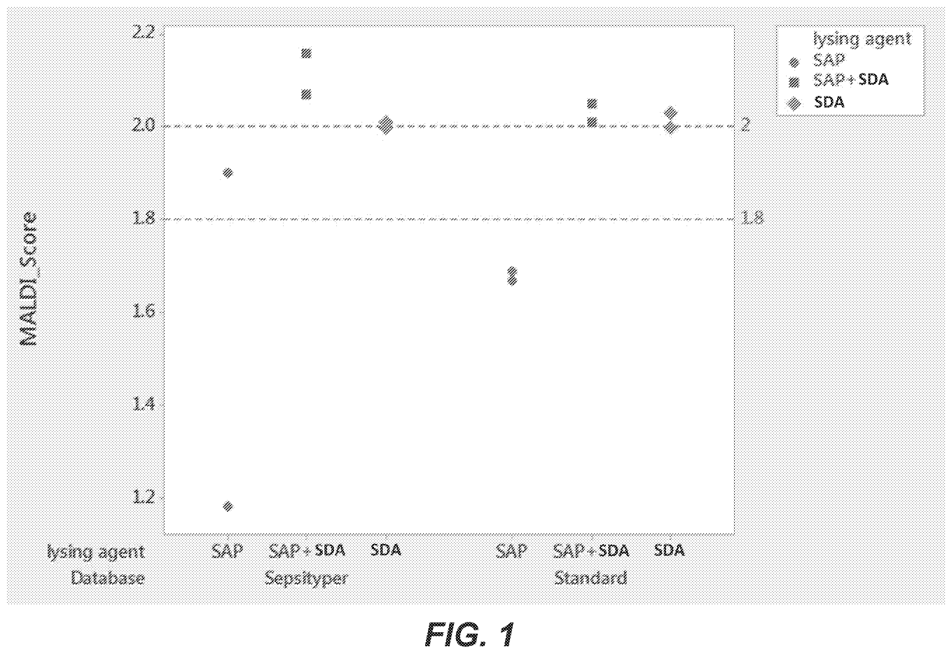

Currently available methods employ saponin to lyse blood cells for isolating bacteria from positive blood cultures. However, MALDI identification on some bacterial strain has very low scores, resulting in no identification, especially for Staphylococcus epidermidis. It is probable that this identification failure is due to the blood cells not being completely lysed and/or a high content of blood debris. SDA was used to replace Saponin in the lysing buffer for removing blood cells and high MALDI scores were obtained with correct identification (FIG. 1). In some embodiments, SDA is very water soluble. An advantage of using SDA as the lysing agent for isolating bacteria from positive blood culture is that the MALDI score for challenging species such as Staphylococcus epidermidis can be higher than the cutoff value, leading to correct species identification. Moreover, processing times using SDA versus saponin can be nearly identical.

There are provided, in some embodiments, methods of processing a sample. In some embodiments, the method comprises contacting a sample comprising blood cells and at least one microorganism with a lysis buffer to generate a treated sample, wherein the lysis buffer comprises a Somatic Cell Digestion Agent (SDA) capable of lysing blood cells in the sample, thereby lysing the blood cells in the sample. There are provided, in some embodiments, compositions (e.g., kits). In some embodiments, composition comprises a lysis buffer comprising a Somatic Cell Digestion Agent (SDA) capable of lysing blood cells, and blood cells and/or debris thereof. In some embodiments, the SDA is a compound of Formula 1:

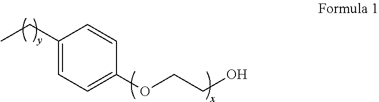

In some embodiments, x is an integer from 2 to 20. In some embodiments, x is an integer from 5 to 15. In some embodiments, x is an integer from 8 to 12. In some embodiments, x is 9 or 10. In some embodiments, x is 9. In some embodiments, wherein y is an integer from 6 to 11. In some embodiments, y is an integer from 8 to 10. In some embodiments, y is 8. In some embodiments, the SDA is Nonoxynol-9. The concentration of the SDA in the lysis buffer, or in the final reaction volume when combined with the sample, can be, can be about, can be at least, or can be at most, 0.001 g/L, 0.005 g/L, 0.01 g/L, 0.05 g/L, 0.1 g/L, 0.2 g/L, 0.3 g/L, 0.4 g/L, 0.5 g/L, 0.6 g/L, 0.7 g/L, 0.8 g/L, 0.9 g/L, 1 g/L, 2 g/L, 3 g/L, 4 g/L, 5 g/L, 10 g/L, 15 g/L, 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, or a number or a range between any of these values. The concentration of the SDA in the lysis buffer, or in the final reaction volume when combined with the sample, can be, can be about, can be at least, or can be at most, 0.001%, 0.005%, 0.01%, 0.05%, 0.1%, 0.2%, 0.3%, 0.4%, 0.5%, 0.6%, 0.7%, 0.8%, 0.9%, 1%, 2%, 3%, 4%, 5%, 10%, or a number or a range between any of these values, (w/w). The concentration of the SDA in the lysis buffer, or in the final reaction volume when combined with the sample, can be about 0.52% (w/w). In other embodiments, the percentages of lysis buffer components disclosed herein are provided as % w/w, % m/v, % v/v, % m/w, % w/v, or variations thereof. The final concentration of the SDA when combined with the sample is not limited so long as the SDA is used at a concentration that will hemolyze (or otherwise break down) at least a portion of the blood cells, while leaving at least a portion of the microorganism(s) in the sample intact and/or viable. The final concentration of SDA when contacted with the sample can be, can be about, can be at least, or can be at most, 0.001%, 0.005%, 0.05%, 0.1%, 0.2%, 0.3%, 0.4%, 0.5%, 0.6%, 0.7%, 0.8%, 0.9%, 1%, 2%, 3%, 4%, 5%, 10%, or a number or a range between any of these values, by volume.

In some embodiments, concentrations of the various constituents of the lysis buffers described herein represent the final concentrations of each constituent in the lysis buffer. In some embodiments, a 1:1 volume ratio of the sample (e.g., PBC) is mixed with the lysis buffer during the contacting step; however, other volume ratios are contemplated. Accordingly, the concentrations of each constituent in the lysis buffer can be adjusted to account for changes in the volume ratio of lysis buffer to PBC sample in order to achieve a desired final concentration of the constituents of the lysis buffer when mixed with the sample (e.g., PBC).

The methods for isolating microorganism(s) from a sample suspected of containing at least one microorganism, for example a PBC sample, described herein can utilize the various lysis buffers comprising a SDA contemplated to rapidly produce a viable microbial pellet that can be used for various downstream testing methods, such as, identification by MALDI-TOF/MS, growth-based phenotypic assays and AST testing. In some embodiments, the method includes adding a portion of a sample with the lysis buffer comprising a SDA to form a mixture. In some embodiments, the volume ratio of sample to SDA-comprising lysis buffer is about 1:1. The mixture can be incubated for a period of time to lyse the blood cells in the PBC sample.

In some embodiments, the ratio of the volume ratio of the sample to SDA-comprising lysis buffer can be, or be about, 1:1, 1:1.1, 1:1.2, 1:1.3, 1:1.4, 1:1.5, 1:1.6, 1:1.7, 1:1.8, 1:1.9, 1:2, 1:2.5, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, 1:200, 1:300, 1:400, 1:500, 1:600, 1:700, 1:800, 1:900, 1:1000, 1:2000, 1:3000, 1:4000, 1:5000, 1:6000, 1:7000, 1:8000, 1:9000, 1:10000, or a number or a range between any two of the values. In some embodiments, the volume ratio of the sample to SDA-comprising lysis buffer can be, or be about, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, 10000:1, or a number or a range between any two of the values.

In some embodiments the sample is contacted with the lysis buffer two or more times. For example, the sample can contacted with the lysis buffer 2, 3, 4, 5, 6, 7, 8, 9, or 10 times during the process of sample preparation. In some such embodiments, the sample can be contacted with the lysis buffer, centrifuged to generate a pellet, the pellet can be resuspended, and then the resuspended pellet subjected to one or more additional contacting steps with the lysis buffer. In some embodiments, the contacting step comprises an incubation period that lasts 0.5, 0.6, 0.7, 0.8, 1, 2, 3, 4, 5, 10, 15, 20, 25, 30, 35, 40, 45, 50, 55, 60, 65, 70, 75, 80, 100, 200, 300, 400, 500, or a number or a range between any of these values, minutes.

Some embodiments of the methods and compositions provided herein are useful for the separation, characterization and/or identification of microorganisms from complex samples such as blood-containing culture media. In some embodiments, the methods disclosed herein allow for the characterization and/or identification of microorganisms more quickly than currently available methods, resulting in faster diagnoses (e.g., in a subject having or suspected of having septicemia) and characterization/identification of contaminated materials (e.g., foodstuffs and pharmaceuticals). The steps involved in the disclosed methods, from obtaining a sample to characterization/identification of microorganisms, can be carried out in a very short time frame to obtain clinically relevant actionable information. In certain embodiments, the disclosed methods can be carried out in less than about 120 minutes, e.g., in less than about 210, 200, 190, 180, 170, 160, 150, 140, 130, 120, 110, 100, 90, 80, 70, 60, 50, 40, 30, 20, 15, 10, 5, 4, 3, 2, or 1 minute, or a range or number between any of these values. In some embodiments, the rapidity of the methods disclosed herein represents an improvement over currently available methods. The disclosed methods can be used to characterize and/or identify any microorganism as described herein. In some embodiments, the disclosed methods can be fully automated, thereby reducing the risk of handling infectious materials and/or contaminating the samples.

Samples that may be tested (e.g., a test sample) by the methods disclosed herein include both clinical and non-clinical samples in which microorganism presence and/or growth is or may be suspected, as well as samples of materials that are routinely or occasionally tested for the presence of microorganisms. The amount of sample utilized can vary greatly due to the versatility and/or sensitivity of the methods disclosed herein. Sample preparation can be carried out by any number of techniques known to those skilled in the art although one of the advantages of the disclosed methods is that complex sample types, such as, e.g., blood, bodily fluids, and/or other opaque substances, may be tested directly utilizing the system with little or no extensive pretreatment. In some embodiments, the sample is taken from a culture. In some embodiments, the sample is taken from a microbiological culture (e.g., a blood culture). In some embodiments, the sample is suspected of, or known to, contain microorganisms therein.

Clinical samples that may be tested include any type of sample typically tested in clinical or research laboratories, including, but not limited to, blood, serum, plasma, blood fractions, joint fluid, urine, semen, saliva, feces, cerebrospinal fluid, gastric contents, vaginal secretions, tissue homogenates, bone marrow aspirates, bone homogenates, sputum, aspirates, swabs and swab rinsates, other body fluids, and the like. In some embodiments, the clinical sample is cultured, and a culture sample is used.

The compositions and methods disclosed herein find use in research as well as veterinary and medical applications. Suitable subjects from which clinical samples can be obtained are generally mammalian subjects, but can be any animal. The term “mammal” as used herein shall be given its ordinary meaning and includes, but is not limited to, humans, non-human primates, cattle, sheep, goats, pigs, horses, cats, dog, rabbits, and rodents (e.g., rats or mice). Human subjects include neonates, infants, juveniles, adults and geriatric subjects. Subjects from which samples can be obtained include, without limitation, mammals, birds, reptiles, amphibians, and fish.

Non-clinical samples that may be tested also include substances, encompassing, but not limited to, foodstuffs, beverages, pharmaceuticals, cosmetics, water (e.g., drinking water, non-potable water, and waste water), seawater ballasts, air, soil, sewage, plant material (e.g., seeds, leaves, stems, roots, flowers, fruit), blood products (e.g., platelets, serum, plasma, white blood cell fractions), donor organ or tissue samples, biowarfare samples, and the like. The methods disclosed herein can be employed for real-time testing to monitor contamination levels, process control, quality control, and the like in industrial settings. In some embodiments, the non-clinical sample is cultured, and a culture sample used.

In some embodiments, samples are obtained from a subject (e.g., a patient) having or suspected of having a microbial infection. In some embodiments, the subject has or is suspected of having septicemia, e.g., bacteremia or fungemia. The sample can be a blood sample directly from the subject. The sample can be from a blood culture grown from a sample of the patient's blood. The blood culture sample can be from a positive blood culture, e.g., a blood culture that indicates the presence of a microorganism. In some embodiments, the sample is taken from a positive blood culture within a short time after it turns positive, e.g., within about 6 hours, e.g., within about 5, 4, 3, or 2 hours, or within about 60 minutes, e.g., about 55, 50, 45, 40, 35, 30, 25, 15, 10, 5, 4, 3, 2, or 1 minute, or a range or number between those values. In some embodiments, the sample is taken from a culture in which the microorganisms are in log phase growth. In some embodiments, the sample is taken from a culture in which the microorganisms are in a stationary phase.

Various embodiments of the disclosed methods can provide high sensitivity for the detection, characterization and/or identification of microorganisms. Various embodiments of the disclosed methods can enable detection, characterization and/or identification without first having to go through the steps of isolating microorganisms by growing them on a solid or semisolid medium, and sampling the colonies that grow. Thus, some embodiments, the sample is not from a microbial (e.g., bacteria, yeast, or mold) colony grown on a solid or semisolid surface.

In some embodiments, the volume of the sample is sufficiently large to produce an isolated sample of microorganisms or a pellet of microorganisms which can be interrogated after the separation/isolation step of the methods disclosed herein is carried out. Appropriate volumes will depend on the source of the sample and the anticipated level of microorganisms in the sample. For example, a positive blood culture will contain a higher level of microorganisms per volume than a drinking water sample to be tested for contamination, so a smaller volume of blood culture medium may be needed as compared to the drinking water sample. In general, the sample size can be less than about 50 ml, e.g., less than about 40, 30, 20, 15, 10, 5, 4, 3, or 2 ml, or a range or number between those values. In some embodiments, the sample size can be about 1 ml, e.g., about 0.75, 0.5, or 0.25 ml, or a range or number between those values. In some embodiments in which the separation is carried out on a microscale, the sample size can be less than about 200 μl (e.g., less than about 150, 100, 50, 25, 20, 15, 10, or 5 μl, or a range or number between those values). In some embodiments (e.g., when the sample is expected to comprise a small number of microorganisms), the sample size can be about 100 ml or more, e.g., about 250, 500, 750, or 1000 ml or more, or a range or number between those values.

The sample can be derived from a blood culture of a subject suspected of having an infection. The sample can comprise a positive blood culture sample determined to comprise at least one microorganism therein. The at least one microorganism can be selected from the group comprising gram-positive bacteria, gram-negative bacteria, and yeast. The at least one microorganism can be S. epidermidis. The at least one microorganism can be Enterococcus faecalis, Pseudomonas aeruginosa, E. coli, and/or S. pneumoniae. The contacting step can comprise sonication, osmotic shock, chemical treatment, or any combination thereof. The lysis buffer can comprise one or more proteinases and/or one or more nucleases. The method can comprise isolating the at least one microorganism from the treated sample to generate at least one isolated microorganism. Isolating the at least one microorganism from the treated sample can comprise separating the at least one microorganism from lysed blood cells. Separating the at least one microorganism from lysed blood cells can comprise: centrifuging the treated sample to produce a pellet and a supernatant; and discarding the supernatant while retaining the pellet comprising at least one isolated microorganism. The method can comprise preparing a plated pure culture from the at least one isolated microorganism and analyzing the microorganism obtained from the plated pure culture. The method can comprise preparing an inoculum from the at least one isolated microorganism and analyzing the at least one microorganism obtained from the inoculum. Methods, apparatuses, compositions, and systems for the isolation and identification of microorganisms from samples (e.g., positive blood cultures) have been described in U.S. Pat. Nos. 10,059,975 and 9,180,448, the content of each is incorporated by reference herein in its entirety.

Some embodiments described herein can be used with the compositions and methods for the rapid processing and identification of microorganisms from positive blood cultures described in U.S. Pat. No. 9,631,221, the content of which is incorporated by reference herein in its entirety. The method can comprise: depositing at least a portion of the pellet comprising at least one isolated microorganism on a surface adapted to be placed in an apparatus configured to determine the identity of the at least one microorganism by mass spectrometry; optionally, drying the deposited sample; treating the deposited sample with a volatile acid solution, wherein the volume percent of the volatile acid can be at least 70% of the volatile acid solution combined with the deposited sample; optionally, drying the treated deposited sample; placing a matrix over the treated deposited sample; and optionally, drying the treated deposited sample. The volatile acid solution can be a volatile acid in water or a volatile solution in an organic solvent. In some embodiments, the volatile acid solution can be formic acid in water at a volume percent of about 60%, of about 65%, of about 70%, of about 75%, of about 80%, of about 85%, of about 90%, of about 95%, or a range or number between those values, when combined with the deposited sample. The method can comprise, prior to treating the deposited sample with a volatile acid solution, treating the deposited sample with an organic solvent and drying the deposited sample. The organic solvent can comprise ethanol, methanol, isopropanol, acetonitrile, acetone, ethyl acetate, or any combination thereof.

Some embodiments described herein can be used with the various reagents (e.g., choline-containing solutions) and methods for rapidly isolating viable microbial cells from positive blood culture samples for use in downstream analyses such as identification and antimicrobial susceptibility testing that have been described in U.S. Pat. No. 8,603,769, the content of which is incorporated by reference herein in its entirety. Although Applicant does not wish to be bound by a particular theory, the addition of a choline-containing solution may inhibit, prevent, and/or mitigate autolysis of the microorganism, in the presence of lytic components (e.g., SDA) of the buffers disclosed herein. The method can comprise contacting the sample with a choline-containing solution before, simultaneously, and/or after contacting the sample with the lysis buffer. In some embodiments, the compositions disclosed herein further comprise a choline-containing solution. The choline-containing solution can comprise at least one quarternary ammonium salt containing a N,N,N-trimethylethanolammonium cation selected from the group consisting of Formula 2,

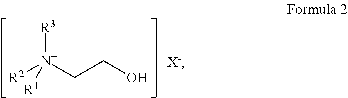

wherein R¹, R², and R³ independently represent one selected from the group consisting of a saturated hydrocarbon group, an unsaturated hydrocarbon group, an aromatic group, and combinations thereof, and wherein X represents a negative charged group. In some embodiments, X is selected from the group consisting of chloride, fluoride, nitrate, and bicarbonate. The choline-containing solution can comprise choline chloride. The choline-containing solution can comprise phosphorylcholine. The final concentration of choline when contacted with the sample can be, can be about, can be at least, or can be at most, 0.001%, 0.005%, 0.05%, 0.1%, 0.2%, 0.3%, 0.4%, 0.5%, 0.6%, 0.7%, 0.8%, 0.9%, 1%, 2%, 3%, 4%, 5%, 10%, or a number or a range between any of these values, by volume. In other embodiments, the percentages of lysis buffer components disclosed herein are provided as % w/w, % m/v, % v/v, % m/w, % w/v, or variations thereof. The concentration of choline in the sample during the contacting can be, can be about, can be at least, or can be at most, 0.001%, 0.005%, 0.01%, 0.05%, 0.2%, 0.3%, 0.4%, 0.5%, 0.6%, 0.7%, 0.8%, 0.9%, 1%, 2%, 3%, 4%, 5%, 10%, or a number or a range between any of these values, by volume. The concentration of choline in the sample during the contacting can be in the range of about 0.25% by volume to about 10% by volume (e.g., 1%, 2%, 3%, 4%, 5%, 6%, 7%, 8%, 9%, 10%, or a range or number between any of these two values). The contacting can comprise incubating the sample with the choline-containing solution for up to 20 minutes. The temperature of said incubation can be room temperature.

The lysis buffers disclosed herein can comprise one or more components to help to stabilize the microorganism(s), lytic reagents lyse the blood cells and/or remove interfering cellular debris. Some embodiments described herein can be used with the various reagents and methods for rapidly isolating viable microbial cells from positive blood culture samples that have been described in U.S. Pat. No. 10,519,482, the content of which is incorporated by reference herein in its entirety. The lysis buffer can comprise an antifoaming agent. In some embodiments, the lysis buffer does not comprise an antifoaming agent. The lysis buffer can comprise at least one thiol. The at least one thiol can comprise L-cysteine HCL, sodium thioglycolate, mercaptoethylamine, mercaptosuccinic acid, mercaptoethanol, mercaptoethane sulfonic acid, thioglycerol, or any combination thereof. The concentration of the at least one thiol in the lysis buffer can be, can be about, can be at least, or can be at most, 0.001, 0.005, 0.01, 0.05, 0.1, 0.2, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 75, 80, or a number or a range between any of these values, g/L. The at least one thiol can comprise L-cysteine and/or sodium thioglycolate. The concentration of L-cysteine and/or sodium thioglycolate in the lysis buffer can be, can be about, can be at least, or can be at most, 0.001, 0.01, 0.05, 0.1, 0.2, 0.3, 0.4, 0.5, 0.6, 0.7, 0.8, 0.9, 1, 2, 3, 4, 5, or a number or a range between any of these values, g/L. The lysis buffer can comprise ammonium chloride. The concentration of ammonium chloride in the lysis buffer can be, can be about, can be at least, or can be at most, 0.001, 0.005, 0.01, 0.05, 0.1, 0.2, 0.3, 0.4, 0.5, 0.6, 0.7, 0.8, 0.9, 1, 2, 3, 4, 5, 10, 20, 25, 30, 35, 40, 45, 50, 55, 60, 65, 70, 75, 80, or a number or a range between any of these values, g/L.

The lysis buffer can comprise a nutrient base solution comprising one or more of casein peptone at a concentration in the lysis buffer of about 8 g/L to about 35 g/L, sodium chloride at a concentration in the lysis buffer of about 2 g/L to about 10 g/L, soy peptone at a concentration in the lysis buffer of about 1.5 g/L to about 15 g/L, potassium phosphate at a concentration in the lysis buffer of about 0.5 g/L to about 5 g/L, and at least one other nutrient. The at least one other nutrient can comprise a nutrient broth. The concentration of the nutrient broth in the lysis buffer can be, can be about, can be at least, or can be at most, 1, 2, 3, 4, 5, 10, 20, 25, 30, 35, 40, 45, 50, 55, 60, 65, 70, 75, 80, or a number or a range between any of these values, g/L. The at least one other nutrient can comprise a nutrient broth comprising one or more of: i) tryptone; ii) soy; iii) NaCl; iv) dipotassium phosphate (K₂HPO₄); and v) glucose. The lysis buffer can comprise one or more of a nutrient broth, an isotonic buffer, a peptone, and a salt. The nutrient broth can comprise trypticase soy broth. The isotonic buffer can comprise sodium phosphate, potassium phosphate, phosphate buffered saline, saline, or any combination thereof. The concentration of the isotonic buffer in the lysis buffer can be, can be about, can be at least, or can be at most, 0.1, 0.2, 0.3, 0.4, 0.5, 0.6, 0.7, 0.8, 0.9, 1, 2, 3, 4, 5, 10, 15, 20, 25, 30, 35, 40, 45, 55, 60, 65, 70, 75, 80, or a number or a range between any of these values, g/L. The peptone can comprise casein peptone and/or soy peptone. The lysis buffer can comprise sodium pyruvate, yeast extract, sodium citrate, meat peptones, dextrose, phosphate buffered saline, or any combination thereof. In some embodiments, the lysis buffer can comprise at least one additional non-ionic detergent (e.g., saponin). In some embodiments, the lysis buffer does not comprise an additional non-ionic detergent.

The method can comprise identifying the at least one microorganism. Identifying the at least one microorganism can comprise mass spectrometry, phenotypic identification, antimicrobial susceptibility testing, molecular testing, or any combination thereof. Mass spectrometry can comprise one or more of electrospray ionization mass spectrometry (ESI-MS), ESI-MS/MS, ESI-MS/(MS)ⁿ, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), APCJ-MS/MS, APCI-(MS)ⁿ, atmospheric pressure photoionization mass spectrometry (APPI-MS), APPI-MS/MS, and APPI-(MS)ⁿ, quadrupole mass spectrometry, Fourier transform mass spectrometry (FTMS), and ion trap mass spectrometry, where n is an integer greater than zero. Mass spectrometry can comprise MALDI-TOF-MS.

In some embodiments, the SDA does not damage the at least one microorganism. The at least one microorganism can remain intact in the presence of the SDA. In some embodiments, the method yields an at least 1% (e.g., 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, 2000%, 3000%, 4000%, 5000%, 6000%, 7000%, 8000%, 9000%, 10000%, or a number or a range between any of these values) higher MALDI score as compared to a comparable method employing a lysis buffer that does not comprise the SDA. In some embodiments, the comparable method employs a lysis buffer comprising saponin. In some embodiments, the lysis buffer selectively lyses at least about 1%, at least about 5%, at least about 10%, at least about 20%, at least about 30%, at least about 40%, at least about 50%, at least about 60%, at least about 70%, at least about 80%, at least about 90%, at least about 95%, at least about 99%, or a number or a range between any of these values, of the blood cells in the sample. In some embodiments, the ratio of blood cells lysed to cells of the at least one microorganism lysed following the contacting step can be, or be about, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, 10000:1, or a number or a range between any two of the values. In some embodiments, the ratio of blood cells lysed to cells of the at least one microorganism lysed following the contacting step can be at least, or be at most, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, or 10000:1. In some embodiments, at least about 50%, at least about 60%, at least about 70%, at least about 80%, at least about 90%, at least about 95%, or at least about 99%, of the cells of the at least one microorganism remain intact and/or viable following the contacting step.

A viable and/or intact microbial pellet resulting from the various embodiments described herein can be used to prepare a common sample for various downstream testing methods including identification by mass spectrometry, for example, MALDI-TOF/MS identification, phenotypic growth-based identification, for example, Phoenix™ ID, and AST testing, for example, Phoenix™ AST testing. In addition, the entire method can be performed in one sample tube without the need for transferring sample between multiple tubes. Therefore, the methods described herein can be readily adaptable to automated systems.

Techniques such as higher PBC sample volume, multiple aliquots of PBC sample, multiple spins, etc., described herein can be deployed to increase the number of microorganism(s) in the starting volume to improve yield. In addition, these methods provide a rapid sample preparation method and are easily automated. Furthermore, the methods and buffers described herein subject the blood cells to lysis, remove interfering substances from the PBC sample, and provide high yields of viable microorganism(s). In one embodiment, the yield of viable microbial pellet can be increased by increasing the starting volume of PBC sample and/or by performing the isolation method on several aliquots from one PBC sample and combining the resulting microbial pellets into one sample.

In one embodiment, the isolated microorganism(s) is processed in preparation for downstream testing. This includes, for example, resuspending at least a portion of the isolated microorganism(s) in a fluid, for example, water, OG, BD Phoenix™ ID broth, or a non-ionic detergent. In one embodiment, the isolated microorganism is prepared for identification by mass spectrometry by resuspending the isolated microbial pellet in a solution and depositing a portion of the resuspended pellet onto a MALDI-TOF MS plate, or by directly depositing a portion of the isolated microbial pellet onto a MALDI-TOF MS plate without first resuspending the pellet in a solution. In another embodiment, the isolated microbial pellet is prepared for BD Phoenix™ ID/AST testing by resuspending the isolated microbial pellet in a solution and adjusting the suspension to a specific concentration of about 0.5 McFarland Standard. Additional methods for preparing the isolated microorganism(s) for downstream analysis are contemplated, known to those skilled in the art, and are not described in detail herein.

The isolated microorganism(s) can be used for multiple downstream analyses, including identification of the microorganism(s) (e.g., mass spectrometry, phenotypical, or molecular identification methods, etc.) and AST testing. The AST methods may be applicable to most manual and automated AST systems known in the art, including BD Phoenix™ ID/AST, disk diffusion (Sensi-Disc), agar dilution, and micro-/macrotube dilution methods. Identification methods and AST testing are well known to one skilled in the art and is not described in detail herein. Additional downstream testing can also include, for example, different phenotypic identification systems or methods utilizing enzymatic, biochemical reactions, different molecular or phenotypic identification systems, and/or growth based identification schemes. They may also be used to detect resistance markers that confer protection of the bacterial isolate from certain antimicrobial agents and classes.

The various methods described herein can further include preparation of a plated pure culture or a single inoculum from the isolated microorganism(s). Methods for the preparation of a plated pure culture or inoculum are known to those skilled in the art. The plated pure culture or inoculum can be prepared to obtain adequate amount of sample should additional downstream testing be required.

A portion of the isolated (e.g., pelleted) microorganism obtained by the disclosed methods can be used to inoculate BD Phoenix™ ID broth (Becton, Dickinson and Company). A portion of the inoculum can be used to inoculate the AST portion of a BD Phoenix™ ID/AST panel (Becton, Dickinson and Company). The BD Phoenix™ ID/AST System is described in, e.g., U.S. Pat. Nos. 5,922,593, 6,096,272, 6,372,485, 6,849,422, and 7,115,384, the contents of which are hereby incorporated by reference in their entirety.

In some embodiments, the lysis buffer does not comprise a buffering agent. In some embodiments, the lysis buffer is acidic. In some embodiments, identifying the at least one microorganism does not comprise spectroscopy (e.g., intrinsic fluorescence spectroscopy). In some embodiments, the method does not comprise density gradient centrifugation. In some embodiments, the lysis buffer does not comprise saponin. In some embodiments, the lysis buffer does not comprise one or more detergents selected from the group consisting of Triton® X-100, Triton® X-100-R, Triton® X-114, NP-40, Genapol® C-100, Genapol® X-100, Igepal® CA 630, Arlasolve™200, Brij® 96/97, CHAPS, octyl β-D-glucopyranoside, saponin, nonaethylene glycol monododecyl ether (C12E9, polidocenol), sodium dodecyl sulfate, N-laurylsarcosine, sodium deoxycholate, bile salts, hexadecyltrimethylammonium bromide, SB3-10, SB3-12, amidosulfobetaine-14, C7BzO, Brij® 98, Brij® 58, Brij® 35, Tween® 80, Tween® 20, Pluronic® L64, Pluronic® P84, non-detergent sulfobetaines (NDSB 201), amphipols (PMAL-C8), and methyl-β-cyclodextrin. In some embodiments, the lysis buffer does not comprise one or more detergents selected from the group consisting of Triton® X-100, Triton® X-100-R, Triton® X-114, NP-40, Igepal CA 630, Arlasolve 200, Brij® 96/97, CHAPS, octyl β-D-glucopyranoside, saponin, nonaethylene glycol monododecyl ether. In some embodiments, the lysis buffer does not comprise one or more detergents selected from the group consisting of sodium dodecyl sulfate, N-laurylsarcosine, sodium dexoychloate, bile salts, hexadecyltrimethylammonium bromide, SB3-10, SB3-12, amidosulfobetaine-14, C7BzO. In some embodiments, the lysis buffer does not comprise one or more detergents selected from the group consisting of Brij® 97, Brij® 96V, Genapol® C-100, Genapol® X-100, and polidocenol. In some embodiments, the lysis buffer does not comprise a polyoxyethylene detergent comprising the structure C₁₂₋₁₈/E₉₋₁₀, wherein C₁₂₋₁₈ denotes a carbon chain length of from 12 to 18 carbon atoms and E₉₋₁₀ denotes from 9 to 10 oxyethylene hydrophilic head groups.

### EXAMPLES

Some aspects of the embodiments discussed above are disclosed in further detail in the following examples, which are not in any way intended to limit the scope of the present disclosure.

### Example 1

### MALDI Scores for Staphylococcus epidermidis Isolated from Positive Blood Cultures Using Different Lysing Agents

This example demonstrates the identification of Staphylococcus epidermidis isolated from positive blood cultures employing the sample processing methods and compositions provided herein. FIG. 1 depicts exemplary data related to MALDI scores for Staphylococcus epidermidis isolated from positive blood cultures using different lysing agents, namely saponin (SAP), Nonoxynol-9 (Somatic Cell Digestion Agent (SDA)), and a combination thereof. The concentration of each lysing agent was used at 0.52% (w/w). The score for acceptance of identification is greater than or equal to 1.8 for the Sepsityper database and 2.0 for the standard database. Surprisingly, the use of SDA in the lysing buffer achieved a higher MALDI score and correct identification. Lower MALDI scores were achieved using saponin, the currently used lysing agent. SDA is generally used as a spermicide by interacting with the lipids in the membranes of the acrosome and the midpiece of the sperm. It was surprising to find that SDA can lyse blood cells very efficiently even though it is generally used as a spermicide.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method for processing a sample, comprising:
contacting a sample comprising blood cells and at least one microorganism with a lysis buffer to generate a treated sample, wherein the lysis buffer comprises a Somatic Cell Digestion Agent (SDA) capable of lysing blood cells in the sample, wherein the SDA is a compound of Formula 1, 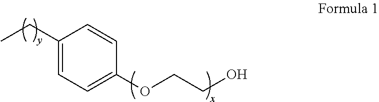
wherein x is an integer from 2 to 20, and wherein y is an integer from 6 to 11, thereby lysing the blood cells in the sample.

2. The method of claim 1, wherein y is an integer from 8 to 10, and optionally y is 8.

3. The method of any one of claims 1-3, wherein x is an integer from 5 to 15, optionally x is an integer from 8 to 12, and further optionally x is 9 or 10.

4. The method of claim 1, wherein the SDA is Nonoxynol-9.

5. The method of any one of claims 1-4, wherein the concentration of the SDA in the lysis buffer is about 0.01 g/L to about 10 g/L.

6. The method of any one of claims 1-5, wherein the concentration of the SDA in the lysis buffer is about 0.01% (w/w) to about 10% (w/w), optionally about 0.01% (w/w) to about 1% (w/w), and further optionally about 0.52% (w/w).

7. The method of any one of claims 1-3, wherein the sample is derived from a blood culture of a subject suspected of having an infection.

8. The method of any one of claims 1-7, wherein the sample comprises a positive blood culture sample determined to comprise at least one microorganism therein.

9. The method of any one of claims 1-8, wherein the at least one microorganism is selected from the group comprising gram-positive bacteria, gram-negative bacteria, and yeast; optionally the at least one microorganism comprises one or more of S. epidermidis, Enterococcus faecalis, Pseudomonas aeruginosa, E. coli, and S. pneumoniae.

10. The method of any one of claims 1-9, wherein the contacting step comprises sonication, osmotic shock, chemical treatment, or any combination thereof.

11. The method of any one of claims 1-10, wherein the lysis buffer comprises one or more proteinases and/or one or more nucleases.

12. The method of any one of claims 1-11, comprising isolating the at least one microorganism from the treated sample to generate at least one isolated microorganism; optionally the isolating the at least one microorganism from the treated sample comprises separating the at least one microorganism from lysed blood cells, and further optionally separating the at least one microorganism from lysed blood cells comprises:
centrifuging the treated sample to produce a pellet and a supernatant; and
discarding the supernatant while retaining the pellet comprising at least one isolated microorganism.

13. The method of claim 12, further comprising preparing a plated pure culture from the at least one isolated microorganism and analyzing the microorganism obtained from the plated pure culture.

14. The method of any one of claims 1-13, further comprising preparing an inoculum from the at least one isolated microorganism and analyzing the at least one microorganism obtained from the inoculum.

15. The method of any one of claims 1-14, further comprising:
depositing at least a portion of the pellet comprising at least one isolated microorganism on a surface adapted to be placed in an apparatus configured to determine the identity of the at least one microorganism by mass spectrometry;
optionally, drying the deposited sample;
treating the deposited sample with a volatile acid solution, wherein the volume percent of the volatile acid is at least 70% of the volatile acid solution combined with the deposited sample;
optionally, drying the treated deposited sample;
placing a matrix over the treated deposited sample; and
optionally, drying the treated deposited sample.

16. The method of claim 15, wherein the volatile acid solution is a volatile acid in water or a volatile solution in an organic solvent; and optionally the organic solvent comprises ethanol, methanol, isopropanol, acetonitrile, acetone, ethyl acetate, or any combination thereof.

17. The method of any one of claims 15-16, wherein the volatile acid solution is formic acid in water at a volume percent of about 70% to about 90% when combined with the deposited sample.

18. The method of any one of claims 15-17, further comprising, prior to treating the deposited sample with a volatile acid solution, treating the deposited sample with an organic solvent and drying the deposited sample.

19. The method of any one of claims 1-18, further comprising contacting the sample with a choline-containing solution before, simultaneously, and/or after contacting the sample with the lysis buffer.

20. The method of claim 19, wherein the choline-containing solution comprises at least one quarternary ammonium salt containing a N,N,N-trimethylethanolammonium cation selected from the group consisting of Formula 2, 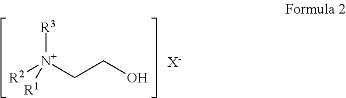
wherein R¹, R², and R³ independently represent one selected from the group consisting of a saturated hydrocarbon group, an unsaturated hydrocarbon group, an aromatic group, and combinations thereof, and wherein X represents a negative charged group.

21. The method of claim 20, wherein X is selected from the group consisting of chloride, fluoride, nitrate, and bicarbonate.

22. The method of any one of claims 19-21, wherein the choline-containing solution comprises choline chloride, phosphorylcholine, or both.

23. The method of any one of claims 19-22, wherein the final concentration of choline when contacted with the sample is greater than or equal to about 0.25% or about 1% by volume.

24. The method of any one of claims 19-23, wherein the concentration of choline in the sample during the contacting is in the range of about 0.25% by volume to about 10% by volume, and optionally about 1.8% to about 4% by volume.

25. The method of any one of claims 19-24, wherein the contacting comprises incubating the sample with the choline-containing solution for up to 20 minutes, and wherein the temperature of said incubation is room temperature.

26. The method of any one of claims 1-25, wherein the lysis buffer further comprises an antifoaming agent.

27. The method of any one of claims 1-25, wherein the lysis buffer does not comprise an antifoaming agent.

28. The method of any one of claims 1-27, wherein the lysis buffer further comprises at least one thiol; optionally the at least one thiol comprises L-cysteine HCL, sodium thioglycolate, mercaptoethylamine, mercaptosuccinic acid, mercaptoethanol, mercaptoethane sulfonic acid, thioglycerol, or any combination thereof; further optionally the concentration of the at least one thiol in the lysis buffer is about 0.005 g/L to 4 g/L.

29. The method of claim 28, wherein the at least one thiol comprises L-cysteine at a concentration in the lysis buffer of about 0.01 g/L to about 2.5 g/L, and/or sodium thioglycolate at a concentration in the lysis buffer of about 0.01 g/L to about 2.5 g/L.

30. The method of any one of claims 1-29, wherein the lysis buffer further comprises ammonium chloride, wherein the concentration of ammonium chloride in the lysis buffer is about g/L to about 80 g/L.

31. The method of any one of claims 1-30, wherein the lysis buffer further comprises a nutrient base solution comprising one or more of casein peptone at a concentration in the lysis buffer of about 8 g/L to about 35 g/L, sodium chloride at a concentration in the lysis buffer of about 2 g/L to about 10 g/L, soy peptone at a concentration in the lysis buffer of about 1.5 g/L to about 15 g/L, potassium phosphate at a concentration in the lysis buffer of about 0.5 g/L to about g/L, and at least one other nutrient.

32. The method of claim 31, wherein the at least one other nutrient comprises a nutrient broth at a concentration in the lysis buffer of about 10 g/L to about 50 g/L.

33. The method of any one of claims 31-32, wherein the at least one other nutrient comprises a nutrient broth comprising one or more of: (i) tryptone; (ii) soy; (iii) NaCl; (iv) dipotassium phosphate (K₂HPO₄); and (v) glucose.

34. The method of any one of claims 1-33, wherein the lysis buffer further comprises one or more of a nutrient broth, an isotonic buffer, a peptone, and a salt, optionally the concentration of the nutrient broth in the lysis buffer is about 10 g/L to about 50 g/L.

35. The method of any one of claims 32-34, wherein the nutrient broth comprises trypticase soy broth.

36. The method of any one of claims 34-35, wherein the isotonic buffer comprises sodium phosphate, potassium phosphate, phosphate buffered saline, saline, or any combination thereof, optionally the concentration of isotonic buffer in the lysis buffer is about 1 g/L to about g/L.

37. The method of any one of claims 34-36, wherein the peptone comprises casein peptone and/or soy peptone.

38. The method of any one of claims 1-37, wherein the lysis buffer further comprises sodium pyruvate, yeast extract, sodium citrate, meat peptones, dextrose, phosphate buffered saline, or any combination thereof.

39. The method of any one of claims 1-38, wherein the lysis buffer further comprises at least one additional non-ionic detergent, optionally the at least one additional non-ionic detergent comprises saponin.

40. The method of any one of claims 1-38, wherein the lysis buffer does not comprise an additional non-ionic detergent.

41. The method of any one of claims 1-40, further comprising identifying the at least one microorganism.

42. The method of claim 41, wherein identifying the at least one microorganism comprises mass spectrometry, phenotypic identification, antimicrobial susceptibility testing, molecular testing, or any combination thereof.

43. The method of any one of claims 15-42, wherein mass spectrometry comprises one or more of electrospray ionization mass spectrometry (ESI-MS), ESI-MS/MS, ESI-MS/(MS)ⁿ, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), APCJ-MS/MS, APCI-(MS)“, atmospheric pressure photoionization mass spectrometry (APPI-MS), APPI-MS/MS, and APPI-(MS)”, quadrupole mass spectrometry, Fourier transform mass spectrometry (FTMS), and ion trap mass spectrometry, where n is an integer greater than zero.

44. The method of any one of claims 1-43, wherein the SDA does not damage the at least one microorganism.

45. The method of any one of claims 1-44, wherein the at least one microorganism remain intact in the presence of the SDA.

46. The method of any one of claims 1-45, wherein the method yields an at least 5% higher MALDI score as compared to a comparable method employing a lysis buffer that does not comprise the SDA.

47. The method of claim 46, wherein the comparable method employs a lysis buffer comprising saponin.

48. The method of any one of claims 1-47, wherein the lysis buffer selectively lyses at least about 1%, at least about 5%, at least about 10%, at least about 20%, at least about 30%, at least about 40%, at least about 50%, at least about 60%, at least about 70%, at least about 80%, at least about 90%, at least about 95%, or at least about 99%, of the blood cells in the sample.

49. The method of any one of claims 1-48, wherein the ratio of blood cells lysed to cells of the at least one microorganism lysed following the contacting step is at least about 2:1.

50. The method of any one of claims 1-49, wherein at least about 50%, at least about 60%, at least about 70%, at least about 80%, at least about 90%, at least about 95%, or at least about 99%, of the cells of the at least one microorganism remain intact and/or viable following the contacting step.

51. The method of any one of claims 1-50, wherein the lysis buffer does not comprise a buffering agent.

52. The method of any one of claims 1-51, wherein the lysis buffer is acidic.

53. The method of any one of claims 41-52, wherein identifying the at least one microorganism does not comprise spectroscopy, and optionally the spectroscopy is intrinsic fluorescence spectroscopy.

54. The method of any one of claims 1-53, wherein the method does not comprise density gradient centrifugation.

55. The method of any one of claims 1-54, wherein the lysis buffer does not comprise:
(a) saponin;
(b) one or more detergents selected from the group consisting of Triton® X-100, Triton® X-100-R, Triton® X-114, NP-40, Genapol® C-100, Genapol® X-100, Igepal® CA 630, Arlasolve™200, Brij® 96/97, CHAPS, octyl β-D-glucopyranoside, saponin, nonaethylene glycol monododecyl ether (C12E9, polidocenol), sodium dodecyl sulfate, N-laurylsarcosine, sodium deoxycholate, bile salts, hexadecyltrimethylammonium bromide, SB3-10, SB3-12, amidosulfobetaine-14, C7BzO, Brij® 98, Brij® 58, Brij® 35, Tween® 80, Tween® 20, Pluronic® L64, Pluronic® P84, non-detergent sulfobetaines (NDSB 201), amphipols (PMAL-C8), and methyl-β-cyclodextrin;
(c) one or more detergents selected from the group consisting of Triton® X-100, Triton® X-100-R, Triton® X-114, NP-40, Igepal CA 630, Arlasolve 200, Brij® 96/97, CHAPS, octyl β-D-glucopyranoside, saponin, nonaethylene glycol monododecyl ether;
(d) one or more detergents selected from the group consisting of sodium dodecyl sulfate, N-laurylsarcosine, sodium dexoychloate, bile salts, hexadecyltrimethylammonium bromide, SB3-10, SB3-12, amidosulfobetaine-14, C7BzO;
(e) one or more detergents selected from the group consisting of Brij® 97, Brij® 96V, Genapol® C-100, Genapol® X-100, and polidocenol; and/or
(f) a polyoxyethylene detergent comprising the structure C₁₂₋₁₈/E₉₋₁₀, wherein C₁₂₋₁₈ denotes a carbon chain length of from 12 to 18 carbon atoms and E₉₋₁₀ denotes from 9 to 10 oxyethylene hydrophilic head groups.

56. A composition, comprising:
a lysis buffer comprising a Somatic Cell Digestion Agent (SDA) capable of lysing blood cells, wherein the SDA is a compound of Formula 1, 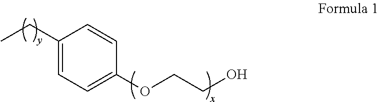
wherein x is an integer from 2 to 20, and wherein y is an integer from 6 to 11; and
blood cells and/or debris thereof.

57. The composition of claim 56, wherein y is an integer from 8 to 10, optionally y is 8.

58. The composition of claim 56-57, wherein x is an integer from 5 to 15, optionally x is an integer from 8 to 12, and further optionally x is 9 or 10.

59. The composition of claim 56, wherein the SDA is Nonoxynol-9.

60. The composition of any one of claims 56-59, wherein the concentration of the SDA in the lysis buffer is about 0.01 g/L to about 10 g/L.

61. The composition of any one of claims 56-60, wherein the concentration of the SDA in the lysis buffer is about 0.01% (w/w) to about 10% (w/w), optionally about 0.01% (w/w) to about 1% (w/w), and further optionally about 0.52% (w/w).

62. The composition of any one of claims 56-61, wherein the lysis buffer comprises one or more proteinases and/or one or more nucleases.

63. The composition of any one of claims 56-62, further comprising a choline-containing solution comprising at least one quarternary ammonium salt containing a N,N,N-trimethylethanolammonium cation selected from the group consisting of Formula 2, 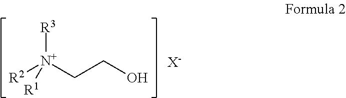
wherein R¹, R², and R³ independently represent one selected from the group consisting of a saturated hydrocarbon group, an unsaturated hydrocarbon group, an aromatic group, and combinations thereof, and wherein X represents a negative charged group.

64. The composition of claim 63, wherein X is selected from the group consisting of chloride, fluoride, nitrate, and bicarbonate.

65. The composition of any one of claims 63-64, wherein the choline-containing solution comprises choline chloride, phosphorylcholine, or both.

66. The composition of any one of claims 56-65, wherein the lysis buffer further comprises an antifoaming agent.

67. The composition of any one of claims 56-66, wherein the lysis buffer does not comprise an antifoaming agent.

68. The composition of any one of claims 56-67, wherein the lysis buffer further comprises at least one thiol; optionally the at least one thiol comprises L-cysteine HCL, sodium thioglycolate, mercaptoethylamine, mercaptosuccinic acid, mercaptoethanol, mercaptoethane sulfonic acid, thioglycerol, or any combination thereof; and further optionally the concentration of the at least one thiol in the lysis buffer is about 0.005 g/L to 4 g/L.

69. The composition of claim 68, wherein the at least one thiol comprises L-cysteine at a concentration in the lysis buffer of about 0.01 g/L to about 2.5 g/L, and/or sodium thioglycolate at a concentration in the lysis buffer of about 0.01 g/L to about 2.5 g/L.

70. The composition of any one of claims 56-69, wherein the lysis buffer further comprises ammonium chloride, wherein the concentration of ammonium chloride in the lysis buffer is about 0.01 g/L to about 80 g/L.

71. The composition of any one of claims 56-70, wherein the lysis buffer further comprises a nutrient base solution comprising one or more of casein peptone at a concentration in the lysis buffer of about 8 g/L to about 35 g/L, sodium chloride at a concentration in the lysis buffer of about 2 g/L to about 10 g/L, soy peptone at a concentration in the lysis buffer of about 1.5 g/L to about 15 g/L, potassium phosphate at a concentration in the lysis buffer of about 0.5 g/L to about 5 g/L, and at least one other nutrient.

72. The composition of claim 71, wherein the at least one other nutrient comprises a nutrient broth at a concentration in the lysis buffer of about 10 g/L to about 50 g/L.

73. The composition of any one of claims 71-72, wherein the at least one other nutrient comprises a nutrient broth comprising one or more of: i) tryptone; ii) soy; iii) NaCl; iv) dipotassium phosphate (K₂HPO₄); and v) glucose.

74. The composition of any one of claims 56-73, wherein the lysis buffer further comprises one or more of a nutrient broth, an isotonic buffer, a peptone, and a salt; optionally the concentration of the nutrient broth in the lysis buffer is about 10 g/L to about 50 g/L; and further optionally the nutrient broth comprises trypticase soy broth.

75. The composition of claim 74, wherein the isotonic buffer comprises sodium phosphate, potassium phosphate, phosphate buffered saline, saline, or any combination thereof, optionally the concentration of isotonic buffer in the lysis buffer is about 1 g/L to about 20 g/L.

76. The composition of any one of claims 56-75, wherein the peptone comprises casein peptone and/or soy peptone.

77. The composition of any one of claims 56-76, wherein the lysis buffer further comprises sodium pyruvate, yeast extract, sodium citrate, meat peptones, dextrose, phosphate buffered saline, or any combination thereof.

78. The composition of any one of claims 56-77, wherein the lysis buffer further comprises at least one additional non-ionic detergent, optionally the at least one additional non-ionic detergent comprises saponin.

79. The composition of any one of claims 56-77, wherein the lysis buffer does not comprise an additional non-ionic detergent.

80. The composition of any one of claims 56-79, wherein the lysis buffer does not comprise a buffering agent.

81. The composition of any one of claims 56-80, wherein the lysis buffer is acidic.

82. The composition of any one of claims 56-81, wherein the lysis buffer does not comprise:
(a) saponin;
(b) one or more detergents selected from the group consisting of Triton® X-100, Triton® X-100-R, Triton® X-114, NP-40, Genapol® C-100, Genapol® X-100, Igepal® CA 630, Arlasolve™200, Brij® 96/97, CHAPS, octyl β-D-glucopyranoside, saponin, nonaethylene glycol monododecyl ether (C12E9, polidocenol), sodium dodecyl sulfate, N-laurylsarcosine, sodium deoxycholate, bile salts, hexadecyltrimethylammonium bromide, SB3-10, SB3-12, amidosulfobetaine-14, C7BzO, Brij® 98, Brij® 58, Brij® 35, Tween® 80, Tween® 20, Pluronic® L64, Pluronic® P84, non-detergent sulfobetaines (NDSB 201), amphipols (PMAL-C8), and methyl-β-cyclodextrin;
(c) one or more detergents selected from the group consisting of Triton® X-100, Triton® X-100-R, Triton® X-114, NP-40, Igepal CA 630, Arlasolve 200, Brij® 96/97, CHAPS, octyl β-D-glucopyranoside, saponin, nonaethylene glycol monododecyl ether;
(d) one or more detergents selected from the group consisting of sodium dodecyl sulfate, N-laurylsarcosine, sodium dexoychloate, bile salts, hexadecyltrimethylammonium bromide, SB3-10, SB3-12, amidosulfobetaine-14, C7BzO;
(e) one or more detergents selected from the group consisting of Brij® 97, Brij® 96V, Genapol® C-100, Genapol® X-100, and polidocenol; and/or
(f) a polyoxyethylene detergent comprising the structure C₁₂₋₁₈/E₉₋₁₀, wherein C₁₂₋₁₈ denotes a carbon chain length of from 12 to 18 carbon atoms and E₉₋₁₀ denotes from 9 to 10 oxyethylene hydrophilic head groups.

83. The composition of any one of claims 56-82, wherein the at least one microorganism remains intact in the presence of the SDA.

84. The composition of any one of claims 56-83, wherein the SDA does not damage the at least one microorganism.
